(19) United States
(12) Patent Application Publication
(10) Pub. No.: US 2003/0101644 A1
(43) Pub. Date: Jun. 5, 2003

(54) Methods of improving winter-spring turfgrass quality

(76) Inventor: George W. Hamilton, JR., State College, PA (US)

Correspondence Address: Pamela A. Ruest, Esq., McQuaide Blasko, 811 University Drive, State College, PA, 16801-6699, US

(21) Appl. No.: 10/270,858

(22) Filed: Oct. 15, 2002

Related U.S. Application Data
(60) Provisional application No. 60/335,987, filed on Nov. 30, 2001.

(51) Int. Cl. A01B 79/02; A01C 1/00; A01G 1/00; A01H 3/00

(52) U.S. Cl. 47/58.1 R

(57) ABSTRACT

A method for enhancing the winter-spring quality of turfgrass by application of a composition containing a sand that has been colored with a dark colorant. The color is typically a dark green or black and can be prepared by mixing a latex paint with a white sand. The darkened sand absorbs radiant sunlight and raises the temperature of the turfgrass thus hastening growth and spring greening. The method is especially useful for golf course putting greens and a preferred application is to apply the composition in the fall prior to snow or ice accumulation. Such use on golf course putting greens will cause the turf to break dormancy and grow earlier than normal in the spring thus enhancing the quality and enjoyment of early spring playing.

### REFERENCE TO RELATED APPLICATION

This application claims the benefit of U.S. Provisional Patent application No. 60/335,987filed Nov. 30, 2001.

### FIELD OF THE INVENTION

The invention relates to the field of turfgrass management. In particular, the invention relates to methods of improving turfgrass quality via application of darkened sand compositions in a specified manner.

### BACKGROUND OF THE INVENTION

As used in this application the term “turfgrass” applies to any maintained natural grass surface including lawns, parks, and sports playing surfaces. More specifically though, the invention is limited to such turfgrass surfaces found in temperate and colder climates having turfgrass species that naturally cycle through a dormancy period due to a cold season. The invention is particular applicable to higher maintenance and higher value surfaces such as golf greens, but all maintained natural grass areas are included in the term.

There are numerous problems that turfgrass managers face in maintaining turfgrass at a standard of quality expected by users. While the problems are many, they can be classified into three primary types: (1) those relating to excessive use, (2) those relating to pests (including disease); and (3) those relating to weather and other environmental conditions.

The primary problems related to excessive use are increased soil compaction and undue turf abrasion. Increased soil compaction reduces the amount of water and air in the soil and reduces the root network of the grass. All of these effects have the result of decreasing the general health and growth rate of the turfgrass. Increased turf abrasion causes uprooted or damaged grass leading to a less dense turfgrass surface. Procedures for addressing these issues related to excessive use are generally well accepted by the industry and include soil aeration techniques, planting of new grass, and increased watering.

Problems relating to pests are numerous and generally well known to those in the field. Some general examples of the types of major pests include weeds, insects, and diseases. The specific pest problems faced by turfgrass managers depend highly on the species of grass and on the location of the turfgrass. Typical solutions to these problems include the application of pesticides and other amendments for controlling weeds, insects and diseases.

The third class of problems includes those resulting from the weather and other environmental conditions. These problems are often the most troublesome to address and are the subject of the present invention. Discolored or damaged turf can result from undesirable weather patterns (e.g. drought, excessive precipitation, and unfavorable temperature patterns). Particularly severe problems can result from low temperatures and precipitation in the form of freezing rain, ice, and snow. Such weather can result in damaged turf or in turf that is undesirable in color or is late to green-up in the spring.

In accordance with its generally accepted meaning, the term “green-up” as used herein, refers to the brief period in spring when grass breaks its dormancy as characterized by a normally brownish color and begins a period of rapid growth including the production of chlorophyll giving it a deep, healthy looking green color. Early green-up is an important issue for sports fields and especially for golf course putting greens as players often associate the quality of a putting green with its color. A deeper and darker green color is generally preferred by golfers. Accordingly, turf managers undertake to “green-up” turf as soon as possible.

Early green-up of golf putting greens is primarily a concern in temperate zones and colder climates where courses are dormant over the winter months and golfers begin playing again in the spring. In such locations golfers typically begin playing before the greens have fully greened-up, and while the danger of a frost or snow is still possible.

Turfgrass managers use several procedures to enhance spring green-up. These include the use of fertilizers and dark organic matter. However, these methods are not completely satisfactory. Fertilizers can be effective in producing a green color but suffer from the disadvantages of cost and the importance of applying the “right” amount. The use of too much fertilizer, especially on delicate golf course putting greens, can lead to damaged turf and to the flourishing of certain pests (weeds and/or diseases). The use of dark organic matter can also be somewhat effective, but tend to lose their sunlight absorbing quality quickly and may not be as uniform as a sand.

To address the problems of ice and snow on turfgrass, managers have tried a number of solutions. These solutions include simply covering the turfgrass with a covering (e.g. a tarpaulin). Tarpaulins are effective in preventing ice and snow from directly covering the grass and are effective in raising the temperature of the grass but they prevent sunlight from reaching the grass and are burdensome to install, remove, and store. The use of ice and snow melting agents (e.g. salts) are also possible but are generally not used by turfgrass managers due to the harm that they can cause to turfgrass.

Despite the options available to turfgrass managers there continues to be a need for improved methods for earlier “green-up” of turfgrass and for limiting the negative effects related to snow and ice covering the grass. These are the needs addressed by the present invention, which relates to the use of darkened sand for improving winter-spring turfgrass quality. Accordingly, the following are selected objects of the invention:

It is generally an object of the invention to improve winter and early spring turfgrass quality.

It is an object of the present invention to provide a material and method for hastening the spring green-up of turfgrass.

Another object of the invention is to cause golf course putting green turf to break dormancy and grow earlier than normal in the spring.

It is also an object of the present invention to prevent or reduce the amount of ice or snow formation on turfgrass.

It is also an object of the present invention to provide a more convenient method for limiting or reducing the negative effects associated with ice and snow deposits on turfgrass.

### SUMMARY OF THE INVENTION

The invention is a method for enhancing the winter-spring quality of turfgrass by application of a composition containing a sand that has been colored with a dark colorant. The darkened sand absorbs radiant sunlight and raises the temperature of the turfgrass and soil helping to break dormancy and thus hastening growth and spring greening. The method is especially useful for golf course greens and a preferred application is to apply the composition in the fall prior to snow or ice accumulation.

### BRIEF DESCRIPTION OF THE DRAWINGS

FIG. 1 shows a graph of color ratings for treated and untreated turfgrass plots according to the fall experiment described below.

FIG. 2 shows a graph of color rating for treated and untreated turfgrass plots according to the spring experiment described below.

FIG. 3 shows a graph of average surface temperatures for treated and untreated turfgrass plots according to the experiment described below.

### DETAILED DESCRIPTION OF THE INVENTION

The use of sand as a topdressing material in turfgrass management is well known. When topdressing, a turf manager applies a light layer of a topdressing material to the desired turf. The topdressing is typically a combination of sand, organic matter and/or soil. Topdressing enhances the turfgrass environment in a number of ways. The benefits include increased thatch control, a smoother playing surface, and improved soil aeration. Putting greens and sports fields especially profit from this maintenance practice, primarily because they are high traffic areas and because of the importance of a smooth and uniform surface.

Additionally, sand (even colored sand) has been used for two other applications related to turfgrass. One of these is the use of a green colored sand to cover bare patches or to hide divots. In this application a handful or two of a sand having a color about the same as that of the turfgrass is placed on a divot or bare spot to hide it as much as possible. The other application of sand to turfgrass of which the inventor is aware is the use of a black sand to melt ice on turfgrass. This application is discussed in Japanese Patent Application No. 10-122589.

While the use of sand as a topdressing material, as a covering material, and for snow or ice melting is known, the present inventor has discovered that a darkened sand can also be used to break the dormancy of a turfgrass earlier in the spring than it would otherwise. The present inventive method results in a turfgrass that breaks dormancy earlier, meaning that the grass begins growing and “greens-up” sooner. This results in a turfgrass that is more usable and more aesthetically appealing, especially in the early spring. This effect is achieved by applying a relatively uniform layer of dark colored sand on the turfgrass. The sand can be applied either in the fall or in the spring and either before or after snow or ice accumulation.

The amount of the sand containing composition that is used in the inventive method can vary considerably but is generally in the range of about 50 to 1500 lbs of composition per 1000 square feet of turfgrass. The amount is preferably between about 100 and 1000 lbs/ft² and most preferably between 200 and 800 lbs/ft². The specific application rate is dependent upon the maintained height of the turfgrass to be treated with higher maintained heights generally requiring a higher application rate. The preferred application rate is a level high enough to produce a change in color of the treated area that is readily noticeable by users, but not so much that the surface is almost completely covered. In one embodiment of the inventive method the amount of sand composition applied is less than the amount that would be used to cover up a bare spot, such as a divot. It is preferred that the composition is spread relatively uniformly throughout the application area. Uniform spreading can be easily accomplished via a commercial spreader commonly used by turfgrass managers.

The color of the sand is important. The sand must be sufficiently dark so that it will absorb most of the sunlight that it is exposed to, thereby raising the temperature of the grass and soil and causing the turfgrass to break dormancy earlier than it otherwise would. The inventor has found that both black and dark green sand work very well in the inventive method, but it is expected that dark blue, yellow and others will work well too. In a preferred embodiment, the sand is either black or a dark green color having a shade that is darker than the green color of the turfgrass to which it is applied.

One way of describing the types of sand that can be used in the present invention is through characteristics found in the Munsell Color System (H. Munsell, A Color Notation, Boston 1905; A. H. Munsell, The Atlas of the Munsell Color System, Boston 1915; F. W. Billmeyer Jr., “Survey of Color Order Systems,” Color Research and Application 12, pp. 173-186 [1987]). In the Munsell Color System all colors are described by three characteristics: hue, value, and chroma. Hue is defined as the quality by which we distinguish one color from another. Value is the quality by which we distinguish a dark color from a light one and by arbitrary definition is given as a number from 0 (black) through 10 (white). The third characteristic, chroma, is the quality that distinguishes the difference from a pure hue to a gray shade. Accordingly, one way of distinguishing the types of sand color useful in the present invention is through the Munsell value characteristic. We prefer sands having a Munsell value of about 5 or less, more preferably about 3 or less.

Many methods for coloring the sand can be used. The main criteria for forming the colored sand is that a dark color is obtained that absorbs most and reflects little of the sunlight. Also, it is extremely important that the materials used are not toxic to turfgrass, which are often quite delicate. Additionally, the colored sand must be compatible with commercial topdressing spreaders. Thus, sands that are sticky would not be acceptable as they would tend to clog the machinery. In accordance with these parameters, the inventor has found that coating the sand with a dark colored latex paint and then drying via standard drying techniques results in the desired properties. Other coloring methods including dyeing and staining are also possible as long as they lead to a darkened sand that sufficiently absorbs sunlight to achieve the aims of the invention—the primary aim being to raise the ground temperature enough to make the turfgrass break dormancy earlier in the spring and hasten grass growth and/or green-up.

The main ingredient of the invention is dark colored sand and in one embodiment of the inventive method the composition contains only the sand. However, additional ingredients commonly used by turfgrass managers can be added to the composition. These ingredients include fertilizers and other growth enhancing substances as well as fungicides and other types of pesticides. If additional ingredients are included, it is preferred that they be no more than 50% of the composition and most preferably no more than 25% or even 10% by weight.

### EXAMPLE

The primary purpose of these examples was to evaluate colored sands as a topdressing material and to determine their affect on causing golf course putting green turf to break dormancy and grow earlier than normal in the spring. Two experiments were conducted on a “push-up” style putting green at the Valentine Turfgrass Research Center in University Park, Pa. The turf was a 60-40 mixture of creeping bentgrass and annual bluegrass, respectively, and maintained at 0.125 inches. In one experiment, sand treatments were applied in late fall and in the other experiment, sand treatments were applied in early spring. Ratings for both experiments were completed in the spring.

The topdressing sands used in the experiments met United States Golf Association (USGA) specifications for putting green construction. The sands were supplied and treated by D.M. Boyd Company of New Wilmington, Pa. The black and green sands were prepared by blending a large quantity of white sand and a black latex paint in a standard blender suitable for mixing aggregates such as sand. The amount of paint used was an amount sufficient to substantially coat all of sand. It is preferred that the coat be relatively thin as thicker coats will increase the drying time required. The sand was removed from the blender and dried with heated air and then screen to provide a uniform size.

In both experiments, plots were 3 by 3 feet and treatments were arranged in a randomized complete block design with three replications. Treatments were applied with a hand-held shaker jar on December 19^{th} and February 18^{th} for the fall and spring experiments, respectively. Black sand was used in both experiments at rates of 400 and 800 lbs/1000 sq ft and dark green sand was also used in the spring experiment at 800 lbs/1000 sq ft. An untreated control and non-colored white sand (the same sand that was dyed black or green) were included as treatments in both experiments.

Turf color was rated on a scale of 1 to 5 with 1 being brown and 5 being dark green, and 3 being acceptable color. Color was rated on February 18^{th} and April 10^{th} for the fall experiment and on April 10^{th} for the spring experiment. Surface temperatures of plots were recorded on April 8^{th}, 10^{th}, and 11^{th} with a Licor infrared meter. Five temperature readings were recorded for each plot and averaged.

Fall Experiment Results

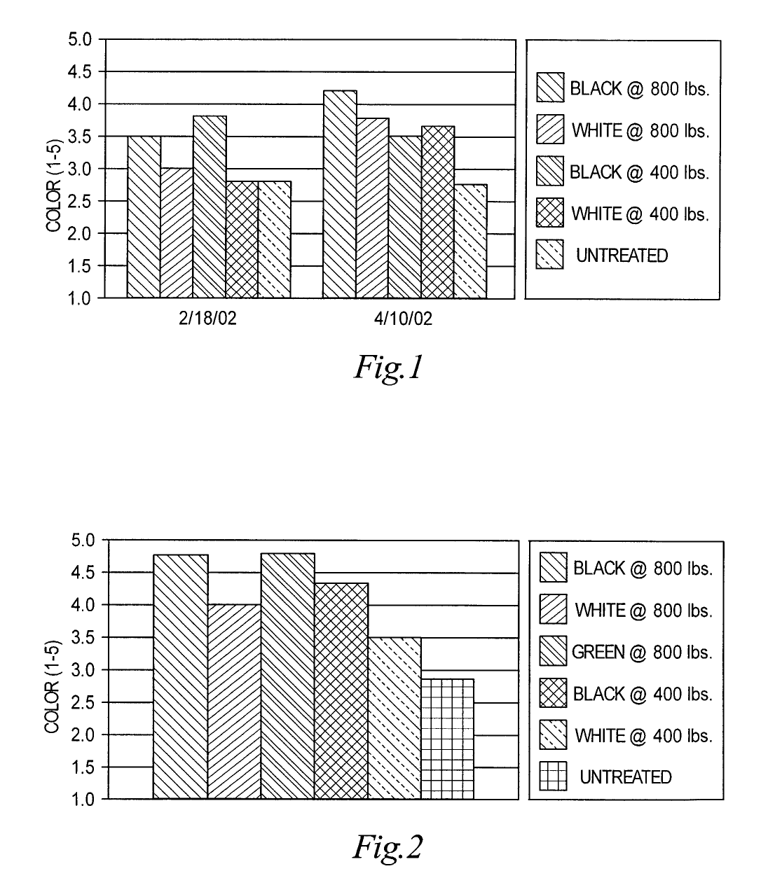

Color rating was done February 18^{th} and April 10^{th}. In February, black sand at 400 and 800 lbs/1000 sq ft provided the best turf green-up with above acceptable ratings of 3.5 and 3.8, respectively (FIG. 1). White sand at 400 lbs/1000 sq ft had the same unacceptable color rating (2.8) as the untreated control. Even at this late winter rating, the black sand at both application rates was able to improve color to an acceptable level. The heavy rate of white sand slightly improved color (3.0) and the low rate of white sand had no effect.

All sand treatments had improved color ratings for the April rating. Black sand at 800 lbs/1000 sq ft had the highest rating (4.3) followed by white sand at 800 lbs/1000 sq ft (3.8). The untreated control color rating (2.7) remained unacceptable and practically unchanged from the February rating. Even though growth had started well before the April rating and the turf was beginning to cover the topdressing, the dyed sand was still effective at improving color. This was probably due to increased soil and turf temperatures resulting from improved absorption of radiant energy caused by the black color. The sand topdressing probably also retained the radiant energy at times of little to no sunlight.

Spring Experiment Results

Black and green sand at 800 lbs/1000 sq ft provided the highest color ratings (4.8) and black sand at 400 lbs/1000 sq ft (4.3) provided a higher color rating than white sand at 800 lbs/1000 sq ft (4.0)(FIG. 2). The untreated control had unacceptable color (2.8) on this rating date. Most of the sand treatments of this experiment had higher color ratings than fall experiment, so the topdressing in the fall experiment plots probably became grown-over and the effectiveness of the colored sand was decreased.

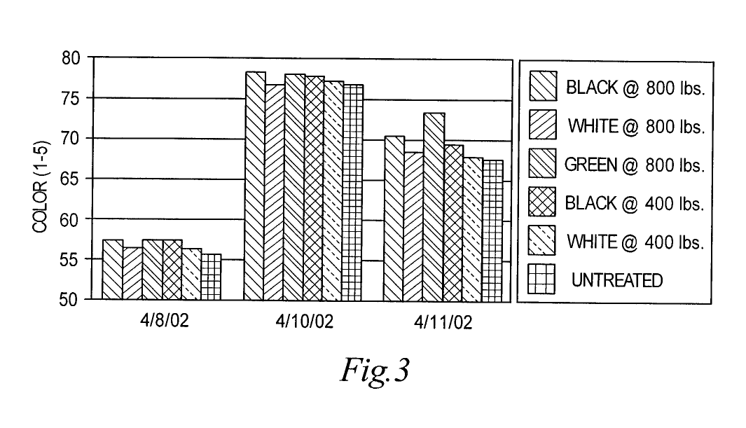

The black and green sand also provided the highest surface temperatures (FIG. 3) on all the rating dates. Black sand at both 400 and 800 lbs/1000 sq ft and green sand at 800 lbs/1000 sq ft had temperatures a degree or two higher than both white sand treatments and the untreated control. A few degree increase in average daily temperature would mimic a multiple week advance of spring time temperatures.

Conclusions

Dark colored topdressing sands increased surface temperatures of putting green turfgrass as compared to white topdressing and an untreated control. Also, surprisingly the dark colored topdressings caused the turf to have improved spring green-up regardless if it was applied in the fall or spring (or prior to or after snow and ice accumulation). Finally, the material application rate did affect topdressing performance with an application of 800 lbs/1000 square feet producing moderately better results than a 400 lbs/1000 square feet application rate.

While the invention has been particularly shown and described with reference to preferred embodiments thereof, it will be understood by those skilled in the art that various alterations in form and detail may be made therein without departing from the spirit and scope of the invention. In particular, the type of sand used, the exact color of the sand, the method of coloring the sand, as well as the specific rate of application may vary widely within the parameters described in this application.

We claim:

1. A method for enhancing the quality of a turfgrass by hastening the breaking of dormancy in the spring, said method comprising applying to said turfgrass a sand containing composition in an amount that is effective at increasing the growth or greening rate of said turfgrass and wherein said sand has been colored with a dark colorant.

2. The method of claim 1 wherein the sand is applied directly to the turfgrass prior to a snow or ice formation.

3. The method of claim 1 wherein the sand is applied after snow or ice accumulation.

4. The method of claim 1 wherein the application rate is an amount that is enough to produce a change in color of the treated area that is readily noticeable but less than an amount that almost completely covers the ground.

5. The method of claim 4 wherein the application rate is between 50 and 1500 lbs/1000 square feet.

6. The method of claim 1 wherein the sand is colored black or green.

7. The method of claim 1 wherein the colored sand has a Munsell value characteristic of 5 or less.

8. The method of claim 7 wherein the colored sand has a Munsell value characteristic of less than about 3.

9. The method of claim 1 wherein the dark colored sand is made by blending the sand with a dark latex paint and then drying.

10. The method of claim 2 wherein the sand is applied in the fall.

11. The method of claim 1 wherein the sand is applied in the spring.

12. The method of claim 1 wherein the composition additionally contains one or more ingredients selected from the group consisting of growth enhancing compounds and pesticides.

13. A method of hastening spring green-up of a golf course putting green turfgrass comprising evenly applying a sand containing composition wherein the sand has been colored with a dark colorant.

14. The method of claim 13 wherein the sand containing composition is applied in the fall prior to the first frost.

15. The method of claim 13 wherein the sand containing composition is applied in the spring.
